(19) United States
(12) Patent Application Publication
(10) Pub. No.: US 2014/0243843 A1
(43) Pub. Date: Aug. 28, 2014

(54) RAPID EXPANSION BALLOON CATHETER

(71) Applicant: Cook Medical Technologies LLC, Bloomington, IN (US)

(72) Inventors: William J. Havel, West Lafayette, IN (US); Steven J. Charlebois, West Lafayette, IN (US); Keith R. Milner, West Lafayette, IN (US)

(21) Appl. No.: 14/139,024

(22) Filed: Dec. 23, 2013

Related U.S. Application Data
(60) Provisional application No. 61/746,933, filed on Dec. 28, 2012.

(51) Int. Cl. A61F 2/958; A61M 29/02

(52) U.S. Cl. CPC A61F 2/958; A61M 29/02; A61F 2002/9586
USPC 606/108; 606/194

(57) ABSTRACT

A balloon catheter assembly including a catheter, a sleeve, and a balloon. The catheter has a body, an exterior surface, a lumen, a port providing fluid communication between the lumen and the exterior surface, and a coupling mechanism to couple the lumen to an inflation source. The sleeve has an interior surface facing the exterior surface, the sleeve being coaxial with the catheter and configured to move longitudinally about the catheter. The balloon is disposed about the catheter and has a middle segment covering the port, a distal segment coupled to the sleeve, and a proximal segment coupled to the catheter. The middle segment has a middle longitudinal stiffness, the distal segment has a distal longitudinal stiffness, and the proximal segment has a proximal longitudinal stiffness. The middle longitudinal stiffness is greater than the proximal longitudinal stiffness and the middle longitudinal stiffness is greater than the distal longitudinal stiffness.

### FIELD

Embodiments of the present invention relate to methods and apparatuses inflating a balloon catheter, and more particularly to a balloon that inflates rapidly.

### BACKGROUND

Balloon catheters are used in a wide variety of medical procedures. For example, they may be used to dilate vessels, expand stents, block fluid flow in a vessel, and anchor a medical device, among other uses.

A balloon catheter typically has at least one lumen extending the length of the balloon catheter. The lumen allows fluid communication between a distal location and the balloon disposed at a proximal end of the balloon catheter. The balloon is inflated by pressurizing a fluid within the lumen causing the balloon to inflate. As the balloon inflates, additional fluid must fill in the increased volume of the inflated balloon. Because the balloon is located at a proximal end of the catheter and a surgeon typically only has access to the distal end of the balloon catheter, the fluid is pressurized at the distal end, and the fluid flows through the lumen to inflate the balloon.

In balloon catheters having a relatively large diameter lumen, the fluid flows relatively easily through the lumen. However, there is a need to expand treatment options to smaller vessels requiring balloon catheters having relatively small diameter lumens. In such balloon catheters the smaller lumen results in increased fluid resistance and the fluid does not flow easily through the lumen. This increased fluid resistance results in a balloon that takes longer to inflate and deflate relative to a larger lumen balloon catheter.

### BRIEF SUMMARY

Embodiments of the invention include a balloon catheter assembly comprising a catheter, a sleeve, and a balloon. The catheter has a catheter body, an exterior surface, a lumen, a port providing fluid communication between the lumen and the exterior surface, and a coupling mechanism to couple the lumen to an inflation source. The sleeve has an interior surface facing the exterior surface, the sleeve being coaxial with the catheter and configured to move longitudinally about the catheter. The balloon is disposed about the catheter and has a middle segment covering the port, a distal segment coupled to the sleeve, and a proximal segment coupled to the catheter. The middle segment has a middle longitudinal stiffness, the distal segment has a distal longitudinal stiffness, and the proximal segment has a proximal longitudinal stiffness. The middle longitudinal stiffness is greater than the proximal longitudinal stiffness and the middle longitudinal stiffness is greater than the distal longitudinal stiffness. The balloon catheter assembly of claim 1 wherein the middle segment has at least one lateral reinforcement.

Another embodiment is directed to a method for deploying a balloon catheter. The method comprises a series of steps. A balloon catheter assembly is provided comprising a catheter having a catheter body and a proximal end, an exterior surface, a lumen, a port providing fluid communication between the lumen and the exterior surface, a sleeve having an interior surface facing the exterior surface, the sleeve being coaxial with the catheter and configured to move longitudinally about the catheter, a balloon disposed about the catheter and having a middle segment covering the port, a distal segment coupled to the sleeve, and a proximal segment coupled to the catheter, wherein the middle segment has a middle longitudinal stiffness, the distal segment has a distal longitudinal stiffness, and the proximal segment has a proximal longitudinal stiffness, wherein the middle longitudinal stiffness is greater than the proximal longitudinal stiffness and the middle longitudinal stiffness is greater than the distal longitudinal stiffness, and an inflation source in fluid communication with the lumen. The proximal end of the catheter is advanced into a body lumen to a treatment site. The balloon is inflated with the inflation source by providing an inflation fluid to the balloon through the lumen and the port. The sleeve is then advanced proximally over the catheter to move the distal segment towards the proximal segment thereby expanding the middle segment radially and folding the distal segment and the proximal segment under the middle segment.

Another embodiment includes a balloon catheter assembly comprising a catheter, a balloon, and a trigger wire. The catheter has a catheter body, an exterior surface, a lumen, a proximal end, and a mechanism adapted to couple the lumen to an inflation source. The balloon is disposed at the proximal end of the catheter, the balloon having an inner volume in fluid communication with the lumen, a distal segment coupled to the catheter, a middle segment, and a proximal segment, wherein the middle segment has a middle longitudinal stiffness, the distal segment has a distal longitudinal stiffness, and the proximal segment has a proximal longitudinal stiffness, wherein the middle longitudinal stiffness is greater than the proximal longitudinal stiffness and the middle longitudinal stiffness is greater than the distal longitudinal stiffness. The trigger wire is coupled to the proximal segment, the trigger wire extending distally along the catheter.

### BRIEF DESCRIPTION OF THE DRAWINGS

To further clarify the above and other advantages and features of the one or more present inventions, reference to specific embodiments thereof are illustrated in the appended drawings. The drawings depict only typical embodiments and are therefore not to be considered limiting. One or more embodiments will be described and explained with additional specificity and detail through the use of the accompanying drawings in which:

FIG. 1 depicts a cross-sectional view of a rapid expansion balloon catheter.

FIG. 2 depicts a cross-sectional view of the rapid expansion balloon catheter of FIG. 1 with the balloon partially expanded.

FIG. 3 depicts a cross-sectional view of the rapid expansion balloon catheter of FIG. 1 with the balloon inflated and compressed longitudinally.

FIG. 4 depicts a plan view of a balloon illustrating the placement of balloon reinforcements.

FIG. 5 depicts the balloon of FIG. 4 being inflated and compressed longitudinally.

FIG. 6 depicts a plan view of a balloon illustrating an alternate placement of the balloon reinforcements.

FIG. 7 depicts the balloon of FIG. 6 being inflated and compressed longitudinally.

FIG. 8 depicts an alternative embodiment having trigger wires for compressing the balloon longitudinally.

### DETAILED DESCRIPTION OF THE DRAWINGS AND THE PRESENTLY PREFERRED EMBODIMENTS

As used herein, “at least one,” “one or more,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C” and “A, B, and/or C” means A alone, B alone, C alone, A and B together, A and C together, B and C together, or A, B and C together.

Various embodiments of the present inventions are set forth in the attached figures and in the Detailed Description as provided herein and as embodied by the claims. It should be understood, however, that this Detailed Description does not contain all of the aspects and embodiments of the one or more present inventions, is not meant to be limiting or restrictive in any manner, and that the invention(s) as disclosed herein is/are and will be understood by those of ordinary skill in the art to encompass obvious improvements and modifications thereto.

Additional advantages of the present invention will become readily apparent from the following discussion, particularly when taken together with the accompanying drawings.

In the following discussion, the terms “proximal” and “distal” will be used to describe the opposing axial ends of the inventive balloon catheter, as well as the axial ends of various component features. The term “proximal” is used in its conventional sense to refer to the end of the apparatus (or component thereof) that is closest to the heart during use of the apparatus. The term “distal” is used in its conventional sense to refer to the end of the apparatus (or component thereof) that is furthest from the heart during use. For example, a catheter may have a proximal end and a distal end, with the distal end designating the end furthest from the heart during an operation, such as a handle, and the proximal end designating an opposite end of the catheter closer to the heart. Similarly, the term “proximally” refers to a direction that is generally away from the heart along the apparatus during use and the term “distally” refers to a direction that is generally away from the heart along the apparatus.

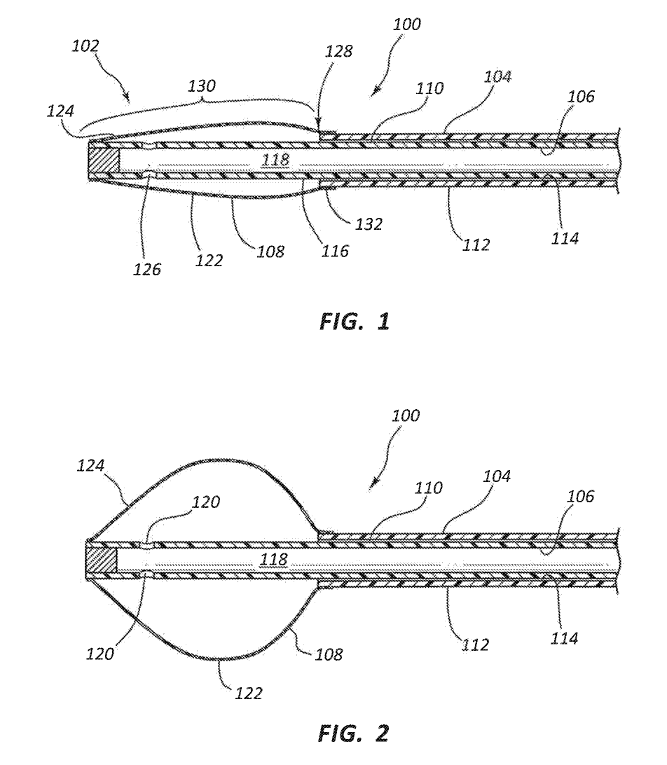

FIG. 1 is a cross-section of a proximal end 102 of an embodiment of a rapid expansion balloon catheter 100. The proximal end 102 is comprised of a sleeve 104, a catheter 106, and a balloon 108. The sleeve 104 has an exterior surface 112 and an interior surface 114 defining at least one lumen 110. The catheter 106 has an exterior surface 116 facing the interior surface 114 of the sleeve 104. The sleeve 104 is coaxial with the catheter 106 and is free to move longitudinally about the catheter 106. The sleeve 104 is constrained from lateral movement by interference between the inner surface 114 of the sleeve and the outer surface 116 of the catheter 106.

The catheter 106 has at least one lumen 118 extending from the proximal end 102 of the rapid expansion balloon catheter 100 to an inflation source (not shown). The lumen 118 provides fluid communication from the inflation source to the proximal end 102. The proximal end 102 of the lumen 118 is typically sealed such that a fluid within the lumen 118 is inhibited from flowing out the proximal end 102. Near the proximal end 102, the catheter 106 has at least one port 120 passing from the lumen 118 to the exterior surface 116 of the catheter 106. The at least one port 120 provides fluid communication from the lumen 118 to the exterior surface 116 of the catheter 106.

The balloon segment 108 is formed of a flexible material 122 suitable for inflation. The catheter 106 extends beyond a proximal end 128 of the sleeve 104 such that a portion 130 of the exterior surface 116 of the catheter 106 is not disposed within the lumen 110 of the sleeve 104. The flexible material 122 wraps around the portion 130 of the exterior surface 116 of the catheter 106 and a proximal segment 126 of the flexible material 122 is adhered to the proximal end 102 of the catheter 106. A distal segment 132 of the flexible material 122 is adhered to the sleeve 104 at its proximal end 128.

A close fit between the exterior surface 116 of the catheter 106 and the inner surface 114 of the sleeve 104 effectively forms a seal that inhibits fluid from traveling between the outer surface 116 and the inner surface 114. In some instances when a close fit is not possible, an actual sealing element may be disposed between the exterior surface 116 and the inner surface 114 to inhibit fluid passing between the two surfaces 116, 114. With the flexible material 122 adhered at its proximal end 102 to the catheter 106 and at its distal end 132 to the sleeve 104, a balloon 124 is formed with the only means of fluid communication into the balloon 134 being through the at least one port 120.

The second lumen 118 is typically filled with a liquid for inflating the balloon. Because a liquid is not readily compressible, any increase in pressure and volume is readily transmitted to the balloon 124 through the at least one port 120. FIG. 2 illustrates the cross section of the rapid inflation balloon catheter 100 with the balloon 124 being partially inflated. The inflation source (not shown) has increased the volume of fluid at the proximal end of the inflation balloon catheter assembly 100 causing the balloon 124 to inflate to accommodate the increased volume of fluid. The balloon 124 may be partially inflated as shown, or it may be inflated further by the inflation source increasing the volume of fluid in the balloon 124. The balloon 124 may inflate to a predetermined diameter for a given volume of fluid.

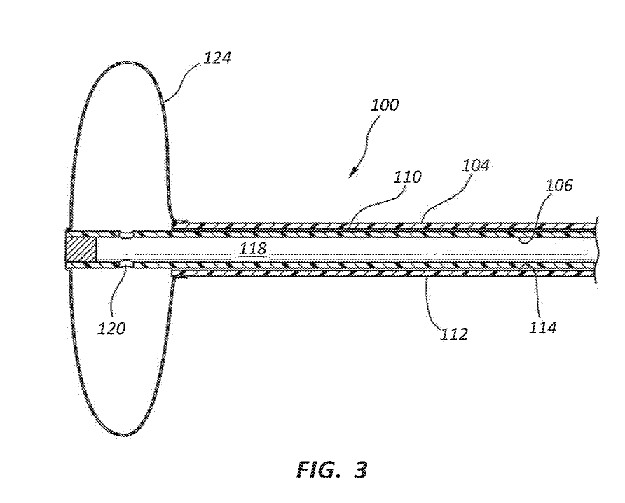

FIG. 3 illustrates the rapid inflation balloon catheter assembly 100 having the catheter 106 retracted axially such that the proximal end 102 of the catheter 106 has moved towards the proximal end of the sleeve 104 compressing the balloon 124 longitudinally. Because the balloon 124 is filled with a liquid that is substantially non-compressible, the reduction in axial length of the balloon 124 results in the balloon 124 expanding radially to maintain the same internal volume prior to the catheter 106g retracted axially. In contrast to inflating the balloon 124 through the inflation source, the axial compression of the balloon 124 may be achieved rapidly since the inflation fluid does not need to flow through the catheter 106 from the inflation source to the at least one port 120. The balloon 124 may expand radially to a predetermined diameter based on the distance the sleeve 104 travels relative to the catheter 106. In some embodiments, the predetermined expanded diameter may be marked on either the sleeve 104 or the catheter 106 such that the balloon 124 can be expanded to a known expanded diameter by moving the sleeve 104 relative to the catheter 106 to one of the marks.

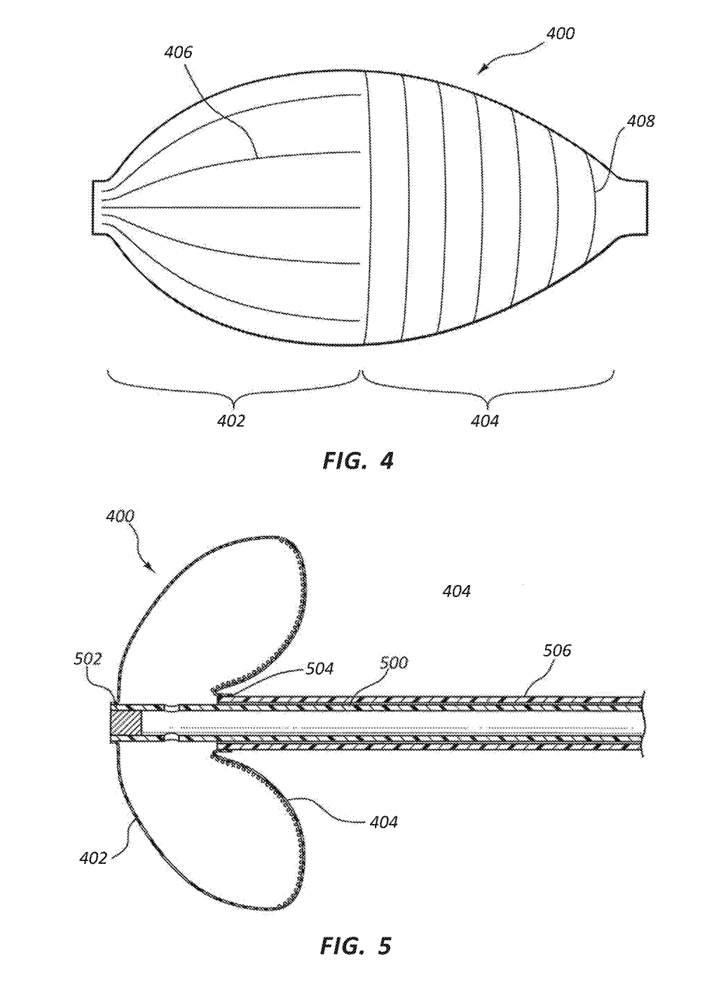

FIG. 4 is a plan view of an example of a partially inflated balloon 400 designed for use with the described rapid inflation catheter assembly 100. The partially inflated balloon 400 is shown without a corresponding catheter and sleeve for the sake of clarity. As described previously, the balloon 400 attaches to a catheter at the balloon's proximal end and to a sleeve at the balloons distal end. The balloon is divided into two segments, a proximal segment 402, and a proximate segment 404. Other numbers of segments are possible and embodiments of the balloon 400 are not limited to this example. The proximal segment 402 has a plurality of longitudinal reinforcements 406 while the distal segment has a plurality of circumferential reinforcements 408, although more segments are possible. The reinforcements vary the stiffness of the balloon 400 in a direction parallel to the reinforcements. For example, longitudinal reinforcements 406 result in an increased stiffness in the longitudinal direction, while circumferential reinforcements 408 increase the stiffness in a circumferential direction. The reinforcements 406, 408 are linear regions in the balloon 400 that have been stiffened, or are stiffer than other regions.

In one embodiment, the reinforcements 406, 408, are made by heat setting the balloon 400 to create folds in the balloon 400, similar to using an iron to create a crease or fold in fabric. In another embodiment, a fiber matrix is incorporated in the balloon with the fiber matrix having the desired shape. In a circumferential reinforcement region the fibers would run around the circumference of the balloon 400 and in the longitudinal reinforcement region, they would run longitudinally along the balloon 400.

FIG. 5 is a schematic showing the general profile of the balloon of FIG. 4 when the proximal end 502 of the catheter 500 is retracted towards the proximal end 504 of the sleeve 506. Because of the longitudinal reinforcements 406, the proximal segment 402 has a greater longitudinal stiffness and resists bending in the longitudinal direction. In contrast, the distal segment's circumferential reinforcements 408 have comparatively little longitudinal stiffness and readily fold under the balloon 400 when compressed.

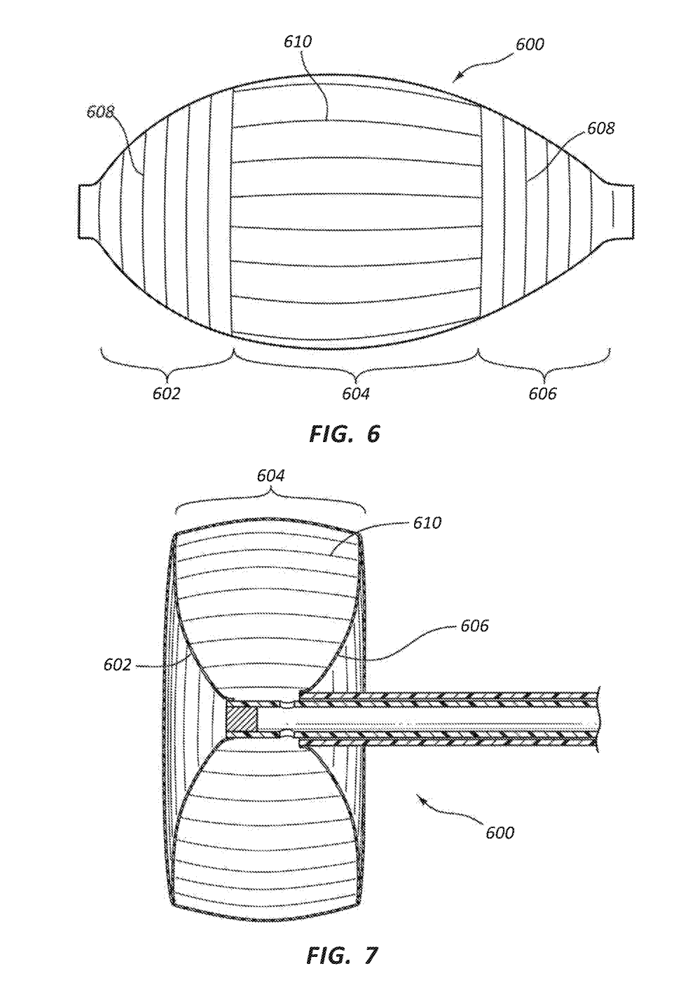

Other configurations of reinforcements are possible. For example, FIG. 6 illustrates a balloon 600 having a proximal segment 602 having circumferential reinforcements 608, a middle segment 604 having longitudinal reinforcements 610, and a distal segment having circumferential reinforcements 608. In this embodiment, when the balloon 600 is compressed longitudinally, the distal segment 606 and the proximal segment 602 roll under the middle segment as depicted in FIG. 7.

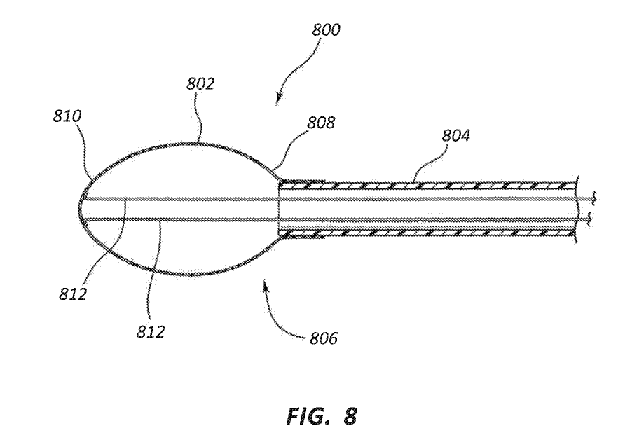

FIG. 8 illustrates another embodiment of a rapid expansion balloon catheter 800. The illustrated embodiment is similar to the previously described embodiment, but differs in the manner of longitudinally compressing the balloon. For the sake of brevity, the foregoing description will not be duplicated and instead, only the structure related to compressing the balloon will be described. In this embodiment, a distal end 808 of a balloon 802 is attached to a catheter 804 at a proximal end 806 of the catheter 804. A proximal end 810 of the balloon 802 is attached to at least one trigger wire 812. The trigger wires 812 extend along the catheter 804 to a trigger mechanism (not shown). The trigger mechanism provides a mechanism for pulling the trigger wires 812 in the distal direction. When the balloon 802 is partially inflated, activation of the trigger mechanism compresses the balloon 802 longitudinally as the proximal end 810 of the balloon 802 is pulled by the trigger wires 812 toward the proximal end 808 of the catheter 804.

The described rapid expansion balloon catheter allows for the balloon to be inflated rapidly in vascular systems requiring the use of a small balloon catheter. The use of the rapid balloon expansion catheter will now be described in relation to FIG. 1.

A surgeon initially guides the proximal end 102 of the rapid expansion balloon catheter 100 to a treatment site. The proximal end 102 is typically guided in a deflated state to minimize the cross section of the proximal end 102. In some embodiments, the proximal end 102 may be guided to the treatment site in a partially inflated state. Once the proximal end 102 is at the treatment site, a fluid is delivered through the second lumen 118 of the catheter 106 to partially inflate the balloon 124.

After the balloon 124 is inflated to a desired size, the fluid delivery path is closed inhibiting the fluid from traveling back distally through the second lumen 118. Because the balloon 124 is only partially inflated and does not occlude the entire vessel at the treatment site, continued proximal perfusion past the balloon 124 is possible.

To rapidly inflate the balloon 124 to occlude the entire vessel at the treatment site, the surgeon advances the sleeve 104 over the catheter 106 bringing the proximal end 128 of the sleeve 104 toward the proximal end 118 of the catheter 106. In one embodiment, the sleeve 104 is advanced by placing the catheter 106 under traction and then pushing the sleeve 104 forward. In another embodiment, the catheter 106 and the sleeve 104 have a threaded engagement which causes the sleeve 104 to advance over the catheter 106 when rotated relative to the catheter 106. In yet another embodiment, the rapid inflation balloon catheter has a sleeve 104 and is advanced through a cannula. A threaded engagement between the cannula and the sleeve 104 causes the cannula to push the sleeve 104 proximally in response to a rotation of the sleeve 104 relative to the cannula.

1. A balloon catheter assembly comprising:
a catheter having a catheter body, an exterior surface, a lumen, a port providing fluid communication between the lumen and the exterior surface, and a mechanism adapted to couple the lumen to an inflation source;
a sleeve having an interior surface facing the exterior surface, the sleeve being coaxial with the catheter and configured to move longitudinally about the catheter; and
a balloon disposed about the catheter and having a middle segment covering the port, a distal segment coupled to the sleeve, and a proximal segment coupled to the catheter, wherein the middle segment has a middle longitudinal stiffness, the distal segment has a distal longitudinal stiffness, and the proximal segment has a proximal longitudinal stiffness, wherein the middle longitudinal stiffness is greater than the proximal longitudinal stiffness and the middle longitudinal stiffness is greater than the distal longitudinal stiffness.

2. The balloon catheter assembly of claim 1 wherein the catheter has a catheter diameter and the middle segment has a balloon diameter, wherein the balloon has a first configuration in which the catheter diameter is substantially equal to the catheter diameter and a second configuration in which the balloon diameter is substantially greater than the catheter diameter.

3. The balloon catheter assembly of claim 1 wherein the middle segment has at least one lateral reinforcement.

4. The balloon catheter assembly of claim 1 wherein the proximal segment has at least one circumferential reinforcement.

5. The balloon catheter assembly of claim 1 wherein the distal segment has at least one circumferential reinforcement.

6. The balloon catheter assembly of claim 1 wherein the proximal segment and the distal segment are adapted to fold under the middle segment in response to the sleeve being advanced proximally over the catheter.

7. The balloon catheter assembly of claim 6, further comprising a stent disposed about the middle segment.

8. The balloon catheter assembly of claim 1 further comprising an inflation source coupled to the lumen.

9. The balloon catheter assembly of claim 1, further comprising a mechanism adapted to advance the sleeve over the catheter.

10. The balloon catheter assembly of claim 9 wherein the mechanism holds the catheter in place while pushing the sleeve.

11. The balloon catheter assembly of claim 9 wherein the mechanism comprises an outside thread disposed on an inner surface of the sleeve and an inside thread disposed on the exterior surface of the catheter, wherein a rotation of the sleeve relative to the catheter causes the sleeve to advance.

12. A method for deploying a balloon catheter comprising:
providing a balloon catheter assembly comprising:
a catheter having a catheter body and a proximal end, an exterior surface, a lumen, a port providing fluid communication between the lumen and the exterior surface;
a sleeve having an interior surface facing the exterior surface, the sleeve being coaxial with the catheter and configured to move longitudinally about the catheter;
a balloon disposed about the catheter and having a middle segment covering the port, a distal segment coupled to the sleeve, and a proximal segment coupled to the catheter, wherein the middle segment has a middle longitudinal stiffness, the distal segment has a distal longitudinal stiffness, and the proximal segment has a proximal longitudinal stiffness, wherein the middle longitudinal stiffness is greater than the proximal longitudinal stiffness and the middle longitudinal stiffness is greater than the distal longitudinal stiffness; and
an inflation source in fluid communication with the lumen;
advancing the proximal end of the catheter into a body lumen to a treatment site;
inflating the balloon with the inflation source providing an inflation fluid to the balloon through the lumen and the port;
advancing the sleeve proximally over the catheter moving the distal segment towards the proximal segment thereby expanding the middle segment radially while folding the distal segment and the proximal segment under the middle segment.

13. The method of claim 12, wherein the body lumen has a treatment sight lumen diameter, and wherein the balloon is inflated to a diameter less than the treatment site diameter prior to advancing the sleeve proximally over the catheter.

14. The method of claim 12 wherein a stent is positioned about the balloon prior to the balloon being advanced into the body lumen, wherein the radial expansion of the middle segment radially expands the stent.

15. A balloon catheter assembly comprising:
a catheter having a catheter body, an exterior surface, a lumen, a proximal end, and a mechanism adapted to couple the lumen to an inflation source;
a balloon disposed at the proximal end of the catheter, the balloon having an inner volume in fluid communication with the lumen, a distal segment coupled to the catheter, a middle segment, and a proximal segment, wherein the middle segment has a middle longitudinal stiffness, the distal segment has a distal longitudinal stiffness, and the proximal segment has a proximal longitudinal stiffness, wherein the middle longitudinal stiffness is greater than the proximal longitudinal stiffness and the middle longitudinal stiffness is greater than the distal longitudinal stiffness; and
a trigger wire coupled to the proximal segment, the trigger wire extending distally along the catheter.

16. The balloon catheter assembly of claim 1 wherein the middle segment has at least one lateral reinforcement.

17. The balloon catheter assembly of claim 1 wherein the proximal segment has at least one circumferential reinforcement.

18. The balloon catheter assembly of claim 1 wherein the distal segment has at least one circumferential reinforcement.

19. The balloon catheter assembly of claim 1 wherein the proximal segment and the distal segment are adapted to fold under the middle segment in response to the trigger wire being sleeve being moved distally along the catheter.

20. The balloon catheter assembly of claim 1 wherein the trigger wire is disposed within the catheter.
